(19) United States
(12) Patent Application Publication
(10) Pub. No.: US 2017/0101611 A1
(43) Pub. Date: Apr. 13, 2017

(54) PHOTOBIOREACTOR IN A CLOSED ENVIRONMENT FOR THE CULTURE OF PHOTOSYNTHETIC MICROORGANISMS

(71) Applicant: ACTA ALGA, Paris (FR)

(72) Inventors: Alain Friederich, Paris (FR); Michel Conin, Paris (FR); Gael Ruiz, Nantes (FR); Mahmoud Affi, Nantes (FR)

(21) Appl. No.: 15/382,198

(22) Filed: Dec. 16, 2016

Related U.S. Application Data
(63) Continuation of application No. 14/115,858, filed on Nov. 5, 2013.

(30) Foreign Application Priority Data
May 6, 2011 (FR) 1153924

(51) Int. Cl. C12M 1/00; C12M 1/34; C12N 1/12

(52) U.S. Cl. CPC C12M 21/02; C12N 1/12; C12M 23/20; C12M 23/22; C12M 31/08; C12M 31/10; C12M 29/02; C12M 41/12

(57) ABSTRACT

The present invention relates to a photobioreactor intended for the notably continuous culture of photosynthetic microorganisms, preferably microalgae, comprising at least one culture enclosure (1) intended to contain the microorganism culture medium (3) and at least one light source (2) outside the culture enclosure (1),
characterized in that it further comprises at least one cylindrical or prismatic light diffusion element (4) placed inside the culture enclosure (1), the light diffusion element (4) being coupled optically with the light source (2) so as to collect the photons emitted by the light source (2) and to return them to the culture medium (3) by its lateral surface.

The present invention also relates to the use of a photobioreactor to cultivate photosynthetic microorganisms and to the use of a light diffusion element (4) to illuminate the culture medium of a photobioreactor.

### GENERAL TECHNICAL FIELD

The invention relates to the intensive and continuous culture of photosynthetic microorganisms.

More precisely, it concerns a photobioreactor intended for one such culture.

### STATE OF THE ART

Microalgae are photosynthetic plant organisms whose metabolism and growth require, among other things, CO₂, light and nutrients.

The industrial culture of microalgae has many applications.

Microalgae can be cultivated to recycle and purify the carbon dioxide, NOx and/or SOx emissions of certain factories (WO 2008042919).

Oil extracted from microalgae can be used as biofuel (WO2008070281, WO2008055190, WO2008060571).

Microalgae can be cultivated for their production of omega-3 and polyunsaturated fatty acids.

Microalgae can also be cultivated to produce pigments.

Traditionally, the large-scale industrial culture of microalgae uses the sun as the light source. To this end, microalgae are often placed in open ponds (raceways) with or without circulation (US2008178739). Other approaches include tube or plate photobioreactors, composed of translucent materials, enabling the passage of light rays in the culture medium in which the microalgae circulate (FR2621323). Other systems comprised of three-dimensional networks of transparent tubes have advantageous space-saving characteristics (EP0874043).

Such installations are extremely voluminous and production yields are low given the uncertainty of sunlight and unproductive night phases, which impede microalgae growth.

In order to reduce size and improve yield, closed photobioreactors were developed. They use the constant (every hour of every day) availability of lighting, which can be interrupted according to sequences specific to the biological cycles of the algae concerned.

Indeed, the crucial factor in increasing microalgae biomass is light, both in terms of quantity and quality since although they absorb all photons of the visible spectrum, microalgae particularly absorb with minimal loss only certain wavelengths of white light.

A photobioreactor is defined as a closed system inside which biomaterial is produced under the action of light energy. Production can be further optimized by controlling culture conditions: nutrients, hydrodynamic medium, gas transfers, liquid circulation rate, etc.

Adapting light, flux and wavelength to the microalgae species is an important factor in optimizing production.

Generally, it is understood that production depends directly on the quality of lighting in the photobioreactor volume. It is necessary that all of the biological liquid is properly illuminated with optimal effective energy. Consequently, the interface between the light sources and the biological liquid must be as large as possible while maximizing the useful volume of the biological liquid.

To clarify these ideas, it will be noted that with concentrations (d) of roughly one gram per liter, light is absorbed at about λ=0.5 cm. For a 1 m³ reactor with a lighting surface of 1 m² (1 m² flat light source), the volume of biological liquid concerned will be only 1/200 m³. The ideal reactor will be one in which the illuminated volume is equal to the reactor volume. More generally, the quality factor of a reactor can be defined by the relationship: Q=Sλ/V₀, wherein S is the illuminated surface (with the proper power) in the volume (V₀) of the reactor, and λ is light penetration depth.

Vₑ being the volume of the lighting elements dispersed in the reactor, production in mass (M) can be expressed by the relationship: M=(V₀−Vₑ)d.

These two relationships must be maximized simultaneously.

Various technological attempts to exploit this dual optimization have been proposed in the past, but they encountered the difficulties described below:

A first artificial lighting solution to solve this problem consists in providing light from a light source in the culture medium near the microalgae using optical fiber (U.S. Pat. No. 6,156,561 and EP0935991).

Optical fiber can be combined with other immersed means to guide light inside the enclosure (JP2001178443 and DE29819259).

The major disadvantage is that this solution only provides low yields (light produced)/(effective light). Indeed, intensity is reduced due to interfaces between the light sources and the waveguide and it is difficult to couple more than one light source to the same fiber. Moreover, a problem arises since several different wavelengths are used: Indeed, for light to leave optical fibers immersed in culture medium the fibers must receive a surface treatment (roughening) to diffuse or diffract a fraction of the guided light. The most effective solution is to etch a network at the periphery of the fiber with a step on the order of the light wavelength carried. This solution has a narrow bandwidth and is completely unsuited when several wavelengths are used. Another artificial lighting solution to solve this problem consists in immersing light sources directly in the photobioreactor enclosure, such as, for example, fluorescent lamps (U.S. Pat. No. 5,104,803) or light-emitting diodes (LEDs) (DE202007013406 and WO2007047805).

This solution improves the energy efficiency of the lighting process because the light sources are closer and better coupled with the culture medium.

However, the use of light sources introduced within the reactor, in particular LEDs, must be carried out while taking into account two other major problems.

The first is inherent to LED emission geometry because their energy emission pattern is directional and follows a Lambertian profile. Only algae within the beam will be lighted. Since the solid angle of the emission cone is typically 90°, three-quarters of the space around an LED will not be lighted. It is noted that the situation will be virtually identical for lighting from the ends of immersed optical fibers.

Moreover, it will be noted that with the LED emission beam being Lambertian, the algae while passing in the transmission beam will receive an inhomogeneous photon flux.

Similarly, when LEDs are used to illuminate an internal wall within the reactor (heat pipe) (see patent DE202007013406) a homogeneous photon flux cannot be obtained in the culture bath.

To attenuate areas in shadow, the light sources within the enclosure could be multiplied and installed sufficiently close to each other.

By doing this a second critical problem arises related to the management of the reactor's thermics which must be controlled within a few degrees, and which depends on the nature of the alga. Indeed, and for typical components such as those currently found on the market, three-quarters of the electric output injected into LEDs dissipates thermically. This thermics management is the second major problem that must be solved. It is inherent in these first-generation reactor structures, independent of the type of light sources used. The dispersion of a large number of light sources in the reactor volume also very quickly poses an electrical connection problem, to which is added the problem of photobioreactor cost if the light sources must be multiplied in great number.

In summary, obtaining a lighting front that is homogeneous in intensity in the reactor's growth volume is a currently unsolved problem. The only way envisaged to approximate a homogeneous front is to multiply the sources inside the reactor, which leads to inextricable problems of heat management.

In order to deal with these problems, the Inventors discovered a novel and particularly effective way to guide and diffuse in the photobioreactor light produced by external LEDs.

Light sources no longer need to be placed inside the enclosure, which greatly facilitates heat regulation. The diffusing light guide used further enables a particularly uniform and homogeneous diffusion of light, and adapts to all wavelengths advantageous to microalgae culture.

### PRESENTATION OF THE INVENTION

Consequently, according to a first aspect, the object of the invention relates to a photobioreactor intended for the culture, notably the continuous culture, of photosynthetic microorganisms, preferably microalgae, comprising at least one culture enclosure intended to contain the microorganism culture medium, and at least one light source outside the culture enclosure, characterized in that it further comprises at least one cylindrical or prismatic light diffusion element placed inside the culture enclosure, the light diffusion element being coupled optically with the light source so as to collect the photons emitted by the light source and to return them to the culture medium by its lateral surface.

According to other advantageous and nonrestrictive characteristics:

the light diffusion element is a solid element made of a transparent material that does not absorb light, at the end of which the light source is placed;

the light diffusion element includes inclusions made of a partially-diffusing material;

the interface between the light source and the light diffusion element is treated with an optical grease that improves photon transmission;

the light diffusion element is a hollow element made of transparent material, at the end of which the light source is placed;

a semi-reflective layer is arranged on the inside of the light diffusion element;

a semi-reflective layer is arranged on the outside of the diffusion element;

the semi-reflective layer (or layers) is made of a metal or metal oxide material, with an optical index that is greater than the index of the material comprising the diffusion element, preferentially aluminum;

the thickness of the semi-reflective layers decreases with distance from the light source;

the light diffusion element is made of poly(methyl methacrylate);

the light source is a quasi-punctual source, and the light diffusion element is a diffusion tube;

the light source is a linear source, and the light diffusion element is a parallelepiped diffuser;

the light source is a light-emitting diode (LED), or a set of LEDs, distributed quasi-punctually or in strips, preferably a high-power light-emitting diode (HPLED), or a set of HPLEDs;

a convergent lens is placed at the interface between the LED and the light diffusion element;

an optical system whose interior is reflective surrounds the LED;

the end of the light diffusion element opposite the light source is provided with a mirror;

the end of the light diffusion element opposite the light source is cone- or dome-shaped;

the external surface of the light diffusion element has suitable roughness to improve light diffusion;

the external surface of the light diffusion element is encapsulated in a protective sheath;

the light diffusion element comprises a cleaning scraper surrounding the sheath;

the photobioreactor comprises a system for cooling the light sources;

the photobioreactor comprises a bubble generation system at the bottom of the culture medium.

A second aspect of the invention relates to the use of a photobioreactor according to the first aspect of the invention, to cultivate photosynthetic microorganisms, preferably microalgae.

A third object of the invention relates to the use of a cylindrical or prismatic light diffusion element coupled optically with a light source so as to collect the photons emitted by the light source and to return them by its lateral surface to illuminate the culture medium of a photobioreactor.

### PRESENTATION OF THE FIGURES

Other characteristics and advantages of the present invention will appear upon consideration of the following description of a preferential embodiment. This description is provided in reference to appended drawings wherein:

FIGS. 1a-d and 2 are diagrams of five embodiments of a light diffusion element of the inventive photobioreactor;

FIG. 3 is a perspective view of a particularly advantageous embodiment of a light diffusion element of the inventive photobioreactor;

FIG. 4 is a perspective view of a parallelepiped embodiment of the inventive photobioreactor;

FIG. 5 is a perspective view of a cylindrical embodiment of the inventive photobioreactor.

FIG. 6 is a perspective view of another parallelepiped embodiment of the inventive photobioreactor.

### DETAILED DESCRIPTION

### Principle of the Invention

Recently, the performance of LED components has greatly improved. There are now high-power LEDs, i.e., more than 10 W of electrical power, which emit at roughly the absorption wavelength of chlorophyll (650 nm-680 nm).

They have in particular optical outputs that exceed industrial products by 25%. In the laboratory, outputs commonly exceeding 35% and in some cases 50% are noted.

This technological breakthrough makes it possible to envisage that a single LED would be sufficient to provide light to a roughly 1-liter volume of culture medium, on the condition of having an optical coupling instrument for diffusing said light.

As a result of research, the Applicant has developed light diffusion elements which collect the light from a light source, in particular from a quasi-punctual or ribbon LED, even placed outside the culture enclosure, and diffuse it in a complete column of culture medium of the photobioreactor.

The fact that the light sources are placed outside the culture enclosure has many advantages, in particular easier heat dissipation, absence of shadows caused by the sources themselves and the ability to maintain electrical connections outside the biological environment, etc.

### Photobioreactor Structure

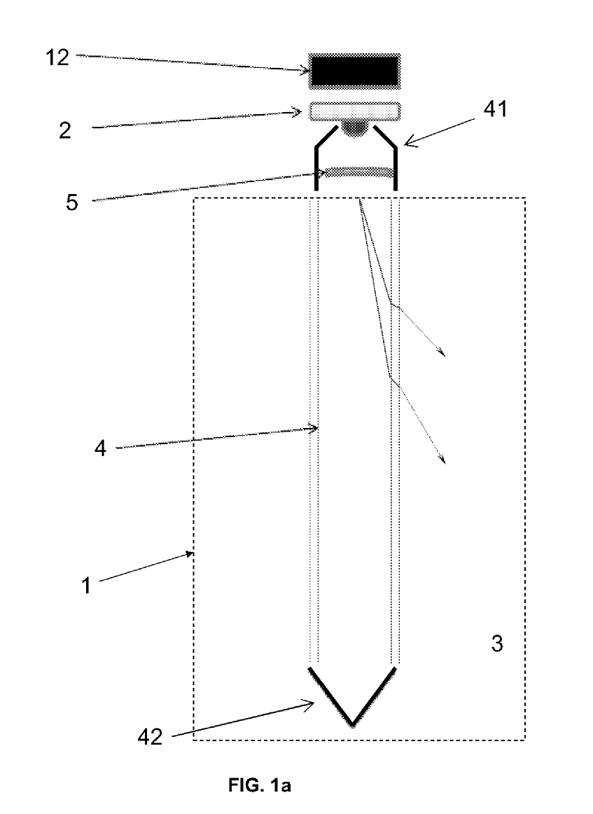

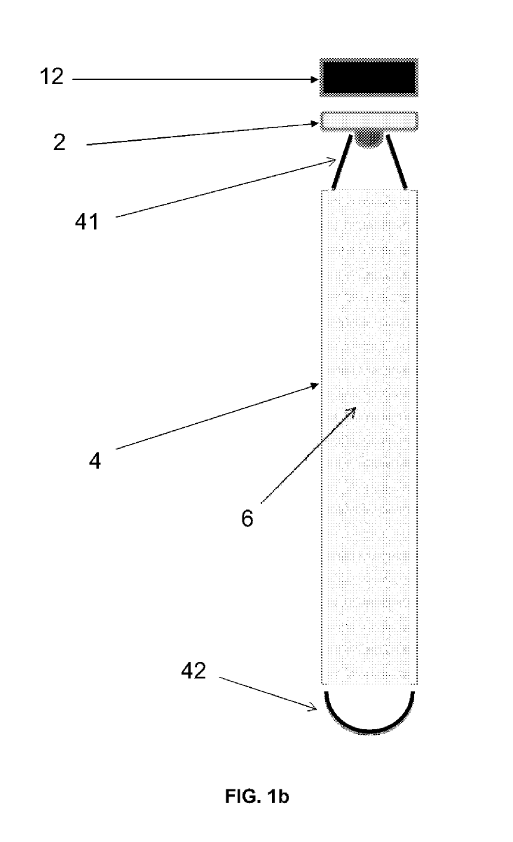

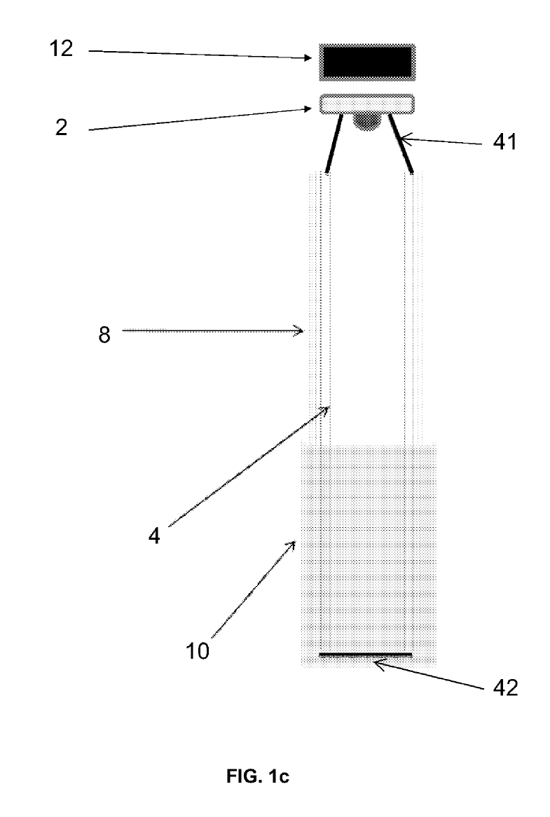

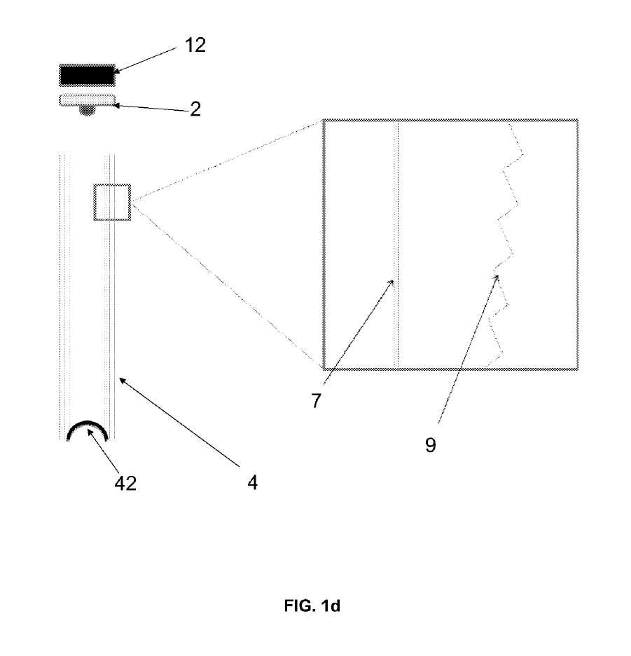

A simplified diagram of a photobioreactor of the invention is presented in FIG. 1a.

This photobioreactor, intended for the notably continuous culture of photosynthetic microorganisms, preferably microalgae, comprises as is seen at least one culture enclosure (1) intended to contain the microorganism culture medium (3), and at least one light source (2) outside the culture enclosure (1).

It further comprises as explained at least one cylindrical or prismatic light diffusion element (4) placed inside the culture enclosure (1), the diffusion element (4) being coupled optically with the light source (2) so as to collect the photons emitted by the light source (2) and to return them to the culture medium (3) by its lateral surface.

In the context of the invention, the following two cases are distinguished: the case in which the light source (2) is a quasi-punctual source, for example a single LED (or a set of single LEDs); and the case in which the light source (4) is a linear source (or a surface), indeed, for example, LEDs in strips or ribbons (see patent application FR1050015).

In both of these cases, a high-power LED (HPLED) (quasi-punctual or ribbon), i.e., an LED of power greater than 1 W, even of power greater than 10 W, is chosen in particular. Hereinafter, the present description will consequently refer primarily to LED light sources, but it will be understood that the invention is in no way limited to this type of source. The person skilled in the art will be able to adapt the inventive photobioreactor to other known light sources (2), including laser sources, which have the advantage of being highly directional, and whose price has dropped considerably.

In all cases, the light sources (2) can be either monochromatic or polychromatic, either naturally or by juxtaposition of monochromatic light sources emitting different wavelengths. It will be noted that it is possible to obtain multispectral LEDs directly by stacking semiconductors of different gaps (including quantum well diodes).

### Geometry of the Light Diffusion Element—Case of Quasi-Punctual Sources

First, it is noted that the emission symmetry of commercial limited LEDs is cylindrical (Lambertian emission), consequently the coupling easiest to carry out is with a tube, whether hollow or solid.

The element (4) is thus referred to as light diffusion “tube” or “finger”. It is however useful to specify that a tube does not necessarily have a circular cross-section, in other words not necessarily a right circular cylinder. The invention relates to any cylindrical or prismatic shape, in other words polyhedrons having a rectangular lateral surface, and on the other hand a constant section, this section having advantageously a central symmetry for respecting Lambertian emission. Indeed, it is certainly possible to envisage diffusion tubes (4) with regular polygonal or star-shaped cross-sections, which would make it possible in particular to increase the lateral surface, i.e., the surface in contact with the microorganism culture medium (3).

A right circular cylinder seems nevertheless the most realistic solution, for reasons of symmetry (diode lobe), and to avoid angular points which would make the luminous front inhomogeneous.

Generally, it should be repeated that the invention is not limited to any geometry, and relates to any cylindrical or prismatic light diffusion element.

Two possibilities of diffusion tubes (4) can be envisaged. According to the first possibility, the diffusion tube (4) is a hollow tube made of transparent material, preferentially of glass or Plexiglas, at the end of which the LED (2) is placed, directed toward the diffusion tube (4) so that the latter receives the photons emitted by the LED (2).

In this configuration, the light is guided in the tube as described in the publication by V. Gerchikov et al. (leukos vol 1 no 4 2005).

In this case light is propagated in the air, i.e., there is no absorption. Given diode divergence (Lambertian), the angles of attack on the inside of the diffusion tube (4) are multiple, and light leaves following a classic law (Descartes law) related to the difference in the index compared to air. The refractive index (n) of air is indeed around 1, and is quite lower than the index (n) of glass or Plexiglas which reaches 1.5. Thus, when an incidental light ray touches the internal surface of the diffusion tube (4), according to its angle of incidence ⊖ in relation to the surface of the tube, the transmission coefficient through the tube passes from almost 1 for an angle of attack ⊖=0° (no propagation) to 0 in the case of low-angle incidence (propagative guidance in the tube). At the interface between the culture medium (3) and the diffusion tube (4) at the lateral surface, nearly the entire luminous flux crosses because the index of water (1.33) is only slightly lower than that of the tube (4). The case described obviously does not relate to the case of a jacketed tube with an air gap. The trajectories of two rays are presented in FIG. 1a. It is supposed that the index of the diffusion tube (4) is close to 1.5.

Advantageously, as is also seen in FIG. 1a, a convergent lens (5) can be placed between the LED (2) and the diffusion tube (4). This lens (5) controls the divergence of the beam from the LED (2). In the single case of a small-aperture injection beam (the diode is in the focal plane of the lens), the majority of the luminous flux is guided. It is understood that by defocusing the beam more or less the luminous flux output of the diffusion tube (4) can be modulated. Correlatively, the penetration depth of the light energy in the diffusion tube (4) can be adjusted to the length of the diffusion tubes. The importance of this point will be seen below.

The injection of light in the hollow diffusion tube (4) can also be improved by surrounding the LED (2) with an optical device (41) for recovering rays at wide angles in relation to the axis of emission to return them in the axis of the tube. There are commercial components that fulfill this function, but they are not suited to the present application considering available space. In the present case, an imperfect but easily carried-out solution is to use a truncated cone whose interior is reflective, with the top of the cone surrounding the LED (2). Several examples of the geometry of one such optical system (41) are presented in FIGS. 1a-c.

According to a second possibility, the diffusion tube (4) is a solid (i.e. non hollow) tube made of a transparent non-light absorbing material, preferentially poly(methyl methacrylate) (PMMA). The index of PMMA (1.49) being the same, with few things near those of water and glass, it will not in principle have guided light if it is plunged in water, but not Fresnel losses at the LED/tube interface (spherical glass encapsulation).

The LED (2) is introduced in a recess made in the diffusion tube (4) (of the size of the spherical segment of the encapsulation of LED (2)).

Advantageous use can be made of a lens (5) which, via the quasi-cylindrical beam that it can produce, enables the light to penetrate in the solid tube (4) (with almost Fresnel losses). The beam thus penetrating in the solid tube (4) in a particularly advantageous way is diffused by the inclusions (6) introduced in the tube. This embodiment is presented in FIG. 1b.

There are indeed industrial systems based on inserting in the mass of PMMA diffusing inclusions (6), i.e., non-absorbent “objects” which ensure the diffusion of light by means of multiple interfaces with random orientations, in particular grains of a material with an index different than that of the tube (4), or air bubbles.

In an even more advantageous manner, the density of the inclusions (6) varies along the height of the diffusion tube (4), and grows with distance from the LED (2) in order to compensate for the progressive loss of light.

The invention is limited to no particular size diffusion tube (4). Said tubes can be up to several meters in length, there is no given limit, and with a diameter most often between a few millimeters and a few centimeters. The diameter is primarily determined by the choice of microalgae concentration in the reactor (continuous mode and/or chemostat) which conditions light penetration and the average power to be applied to the microalgae. These dimensions will be discussed below.

### Geometry of the Light Diffusion Element—Case of Linear Sources

As explained above, the use of tubular diffusion elements (4) to diffuse light is not the only possible configuration. Linear and LED ribbon light sources (2) can indeed be used. It is noted as specified above that the LED ribbon can be composite (several wavelengths) or with polychromatic construction.

In this case, the diffusion elements (4) are advantageously more or less parallelepiped in order to take into account the emission geometry of a ribbon of LEDs. It is noted that it is prismatic geometry in this particular case.

One such a parallelepiped light diffuser (4) is presented in FIG. 2. It can be solid or hollow and can be the subject of the same embodiments as the tubular elements. The present description refers below to “light diffusion tubes”, but it will be well understood that all the possibilities that have been and will be described in the present description (structures, treatments, materials, etc.) can apply as well regardless of the geometry of the diffusion element (4), whether tube or parallelepiped.

### Surface Treatments—Semi-Reflective Treatments

To illuminate the culture medium (3) in an as homogeneous as possible manner, it should be made so that the light exits the diffusion tube (4) with a constant intensity along the light guide, in particular by preventing light from leaving the diffusion tube (4) too early.

In the case of a hollow diffusion tube (4), this light containment effect can be increased advantageously by arranging a semi-reflective layer (7) on the inside of the diffusion tube (4), comparable to a semi-mirror.

In all the diffusing tubes, another semi-reflective layer (8) can be arranged on the outside of the diffusion tube (4), including hollow tubes by replacing or supplementing an internal layer (7).

These internal/external surface treatments, an example of which is seen in FIG. 1c, make it possible to better guide the light.

It is in this case a semi-reflective treatment which can be typically obtained with a metal or metal oxide material, with an optical index that is greater than the index of the material comprising the diffusion tube (4), preferentially aluminum. By increasing the index, reflection is favored over transmission. The quality of the coating is related essentially to its absorption, which must be minimal. Available in the arsenal are semi-transparent optical layers and optical multi-layers (metals or oxides) for fulfilling this function of increasing the mirror effect, which can be adapted to the wavelength of the light used.

Putting a semi-reflective layer (8) on the exterior of the finger, in the case of a hollow tube, is not a necessity, but it simplifies the technique for depositing the semi-reflective material. It can, however, be envisaged to proceed with deposition by soaking in a bath that covers both the outside and the inside of the tube. The semi-reflective layers (7, 8) can be deposited more generally by any chemical (soaking), electrolytic, cathode sputtering, chemical vapor deposition (CVD) or evaporation method, etc.

The materials envisaged are as explained from metals (Al, Ag, etc.) which make it possible to constitute semi-transparent layers of small thickness (nanometer to a few microns) to transparent oxides (Indium doped or not, rare earth, etc.) to fulfill this function. In the transparency ranges necessary herein, the intrinsic absorption of this layer should not exceed 10%.

Even more advantageously, the thickness of the semi-reflective layers (7, 8) decreases with distance from the LED (2), so as to compensate for the progressive loss of light. The person skilled in the art will be able to select the thickness variation profile of the semi-reflective layers (7, 8) (as a function of distance to the LED (2)) to optimize (equalize) the light energy leaving the tube (4). Here again is the same concern that leads to having a variable density of inclusions (6) in the case of a solid diffusion tube (4) (see above). For example, a layer of aluminum whose thickness varies from 20 nm to 100 nm is advantageous.

### Surface Treatments—Diffusing Treatments

It has been seen that certain surface treatments amplify the mirror effect inside the diffusion tube (4), but other treatments make it specifically possible to improve light diffusion.

Thus, advantageously the external surface of the diffusion tube (4) has an increased roughness (9) that improves light diffusion. Adapted roughness refers in particular to roughness on scales comparable to or greater than the wavelength of the light used.

This is, for example, roughness obtained by abrasion, chemical attacks, molding in the vicinity of the softening temperature of PMMA, or by laser etching, etc. The first treatment (semi-reflective) and this second treatment can used separately or simultaneously, for example by depositing a semi-reflective layer (8) on a diffusion tube (4) made rough, making it possible to optimize the flux of light from the diffusion tube (4). A diffusion tube (4) in which a roughness (9) and a semi-reflective internal layer (7) are combined is presented in FIG. 1d.

As with the other treatment, the level of roughness can increase while moving away from the LED (2) to compensate for the loss of luminous flux further away from the source. The optimization of this progressive loss of flux in the light diffusion tube (4), as well as the optimization of output flux constancy when traveling along the diffusion tube (4), aims at the near-total attenuation of light over twice the length of the diffusion tube (4) (no luminous power returning to the source). Thus, advantageously, the end of the diffusion tube (4) opposite the LED (2) is provided with a mirror (42).

At mid-distance (length of the diffusion tube (4), since the complete path is a round-trip), light is returned, which makes it possible to compensate for the loss of light extracted from the tube when moving away from the LED (2) on the “outbound” journey. This mirror can be advantageously tilted according to a predetermined angle or even formed, for example by taking a conical form (as seen in FIG. 1a). Various examples of mirror (42) geometries are also visible in FIGS. 1a-d. It is noted that the use of semi-reflective layers (7, 8) of variable thickness according to the distance from the LED (2) constitutes an additional degree of freedom in optimizing light extraction.

It is further noted that to take into account reactor hydrodynamics (flow of water and bubbles), the end of the diffusion tube (4) opposite the LED (2) is advantageously in the shape of cone or dome to facilitate the flow of water or bubbles (in sparging zones), as will be seen below. If a double-walled tube is used, it is the end thereof which must be shaped into a cone or dome.

### Other Improvements of the Diffusion Tubes

In a preferred manner, the external surface of the diffusion tube (4) is encapsulated in a protective sheath (10). Encapsulation plays the essential role of protecting in particular the semi-reflective layer (8) of the culture medium (3) which by nature is corrosive.

If the external surface of the diffusion tube (4) is artificially rough (9), it is noted that it increases microalgae attachment, which is why it is also desirable to encapsulate the diffusion tube (4).

The protective sheath (10) should be made with a smooth and transparent material (for example plastics again like PMMA, polycarbonate, crystal polystyrene, etc.) on which algae attachment is as weak as possible.

In the case of roughness (9), it is noted that it is necessary to create an index break on the passage of light to obtain the roughness diffusion effect. Thus it is necessary either to choose for the sheath (10) a material with a low index such as polytetrafluoroethylene, or to envisage in a preferred manner an air gap between the sheath (10) and the highly-rough (9) diffusion tube (4), the distance to be crossed by the light in the air having to be advantageously much greater than the degree of roughness (9) (at least a factor of 10).

Generally, the invention will not be limited to any particular embodiment, and could be the object of any possible combinations of semi-reflective layers or roughness on the outside and/or inside if one exists. It is also possible to combine several materials in particular with different indices, and to assemble said various materials into concentric multi-layers. The person skilled in the art will be able to adopt all these options according to the production characteristics selected for the photobioreactor (algae concentration, diffusion tube (4) density, desired yield, desired cost, etc.).

It will be seen below that the sheath (dual tube or encapsulation) makes it possible to envisage an external light tube cleaning system.

### Cooling System

The HPLEDs used preferentially have as explained an output of roughly 25%, i.e., 75% of the power supplied is dissipated in heat.

In other words, use of the LEDs (2) requires the evacuation of significant heat, which is why the photobioreactor comprises advantageously an LED (2) cooling system (12).

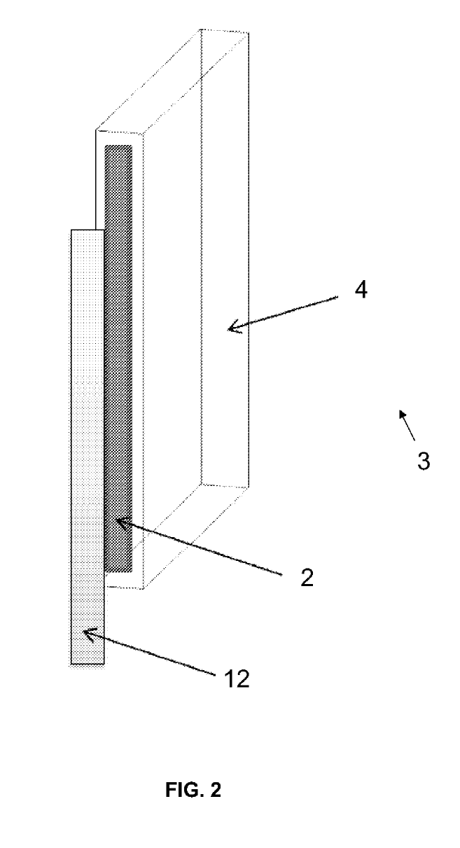

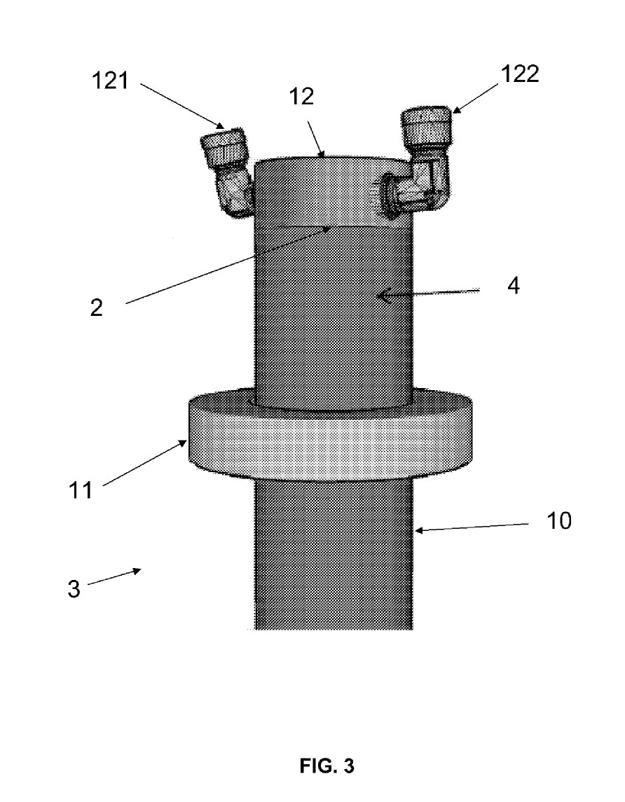

The LEDs (2) for example are assembled on a metal support a few centimeters square which will be placed in direct contact with the cooling system (12), called a heat pipe, comprised of two metal plates between which is circulated a liquid of high thermal conductivity, pulsed air, water or other. Individual radiators cooled by air or water, as seen in FIG. 3, can also be envisaged. Elements (121) and (122) correspond, respectively, to coolant inflow and outflow. In the case of individual radiators, it can be envisaged to connect them in series and/or parallel. Coolant flow rate is controlled by temperature measured at the base of the LEDs.

The LED (2) is in this case assembled on a base at the top of the diffusion tube (4), and is in contact with its heat pipe (12). Its spherical emissive side is in contact with the light diffusion tube (4) (a spherical hole is made if the diffusion tube is solid, the hole advantageously being filled with optical grease).

Alternatively, if it is desired to displace by a few centimeters the LEDs and their electrical connections from the culture medium, a loss-free light guide (cylindrical mirror) of a few centimeters in length can be used at the end of the diffusion tube (4). This guide can be, for example, a truncated cone whose interior is covered with a mirror.

### Cleaning Scraper

In envisaging a protective sheath (10), it is probable that algae will adhere to it. It is thus advantageous to envisage a cleaning system, which is why the diffusion tube (4) advantageously comprises a cleaning scraper (11) surrounding the sheath (10).

The cleaning scraper (11), also visible in FIG. 3, consists for example of a rubber O-ring surrounding the diffusion tube (4) at its upper part. When the diffusion tube (4) is withdrawn (by pulling by the top) the joint scrapes away the algae deposits.

### Photobioreactor Geometry

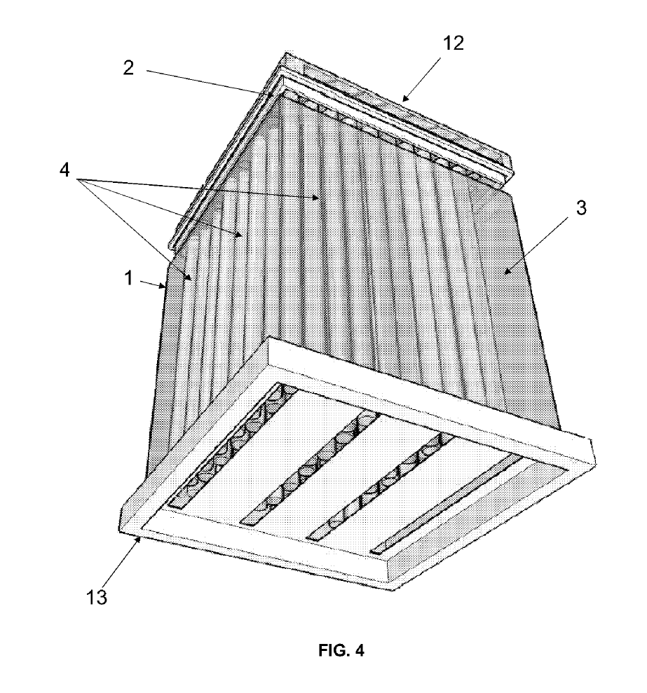

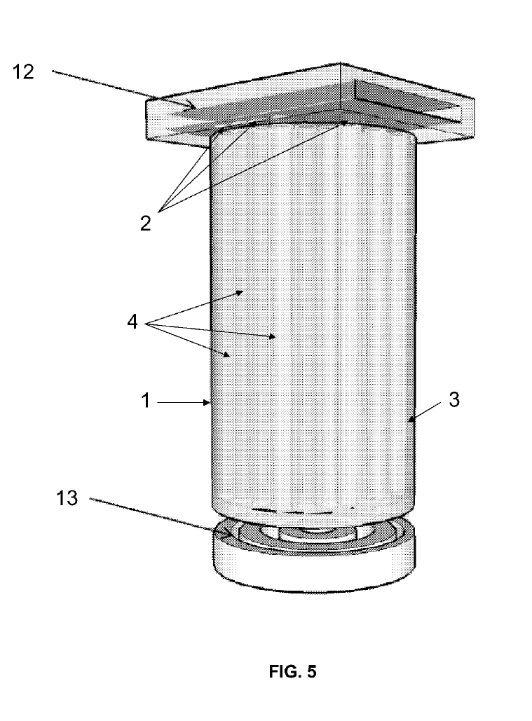

The size of a culture enclosure (1) of the photobioreactor can be quite variable, ranging from a few liters to hundreds of cubic meters. The general geometry of a culture enclosure (1) is generally parallelepiped (FIG. 4) or cylindrical (FIG. 5), but has no or little effect, except possibly with regard to boundary effects and construction costs, on pressure resistance. The photobioreactor can further comprise only one or many culture enclosures (1); the invention is limited to no size or geometry.

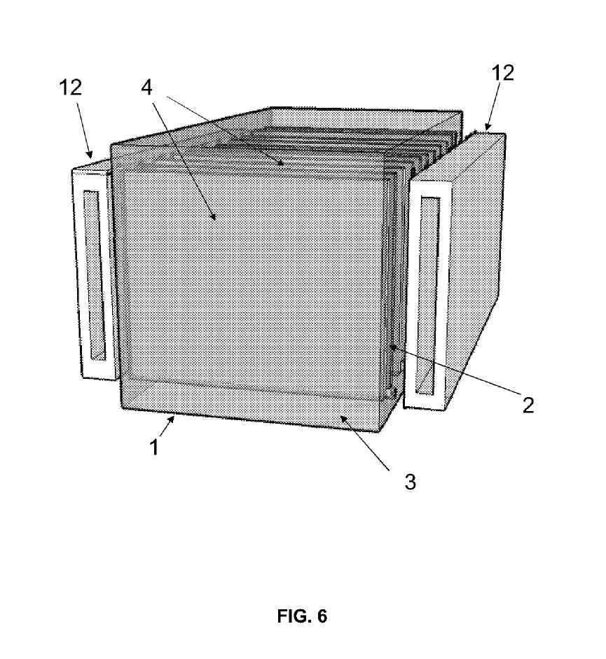

In the case of parallelepiped light diffusers (4), the culture enclosure is preferentially also parallelepiped, as seen in FIG. 6. It is noted that in this example the light sources (2) (and thus the heat pipes (12)) are placed on the sides of the photobioreactor, a symmetrical configuration that increases the flux of light in the guides, but is not absolutely necessary. On the other hand it makes it possible to easily illuminate with two different wavelengths.

The description continues with, as an example, a photobioreactor comprising a single cubic culture enclosure (1) in conformity with FIG. 4, with a total volume of 1 m³ (volume of culture medium (3) plus volume of diffusion tubes (4)).

As seen in FIG. 4, the selected light diffusion tubes (4) described above are approximately 1 m in length in order to illuminate the entire height of the culture enclosure (1) and are optimized to emit a constant flux along their entire height. If the light sources had been lateral, the width of the culture enclosure would have to be considered.

Arrangement of the diffusion tubes (4) in the culture enclosure volume (1) aims to optimize overall homogenization of the flux of light emitted in the culture medium (3). The dimensioning parameter for having a “bath” of light that is near-homogeneous in intensity is the “effective penetration depth” of the light (λ_{eff}).

This parameter is defined from the “characteristic penetration depth” (λ), mentioned in the introduction, which is the length of the culture medium at the end of which a luminous incidental flux is divided by e=2.71828, and a light intensity threshold (I_{eff}) called the “production cycle trigger threshold”, which includes activation of the Calvin cycle. The Calvin cycle is indeed a series of biochemical reactions which take place in the chloroplasts of organisms during photosynthesis. This trigger threshold, expressed in moles of photons per m² per second, corresponds to the minimum level of luminous flux to prime biomass production by the microorganisms. It is typically 50 μmol/m⁻²/s⁻¹ of “red” photons (wavelength around 650 nm) for microalgae (for example of the genus Nannochloris).

For information purposes, a photosynthesis saturation threshold is also found, above which biomass production speed increases no further and even decreases at strong intensities by the destruction of the microalgae.

λ_{eff} is defined as the distance beyond which the luminous flux falls below the threshold I_{eff}.

The Beer-Lambert law enables us to express luminous flux at a distance x of a light source producing an incidental luminous flux I₀: I(x)=I₀e^{-x/λ}.

$$From which {I}_{eff} = {I}_{0} {e}^{- \frac{λ eff}{λ}} , a nd {λ}_{eff} = λl n ( \frac{{I}_{0}}{{I}_{eff}} ) .$$

λ_{eff} is inversely proportional to microalgae concentration, and at a fixed concentration it is determined by the microalgae species. It is considered that a point located at a distance from a light source beyond λ_{eff} does not receive sufficient photons to produce organic matter. In other words, this means that each point of the culture medium (3) must be on average at a distance less than λ_{eff} from the diffusion tube (4). The average distance between two tubes is thus advantageously on the order of 2λ_{eff}.

Adopting this approach, a first possible configuration consists in creating a square network of diffusion tubes (4). While supposing as an example that the tube diameter is d=λ_{eff}=10 mm, a 1 m³ cubic culture enclosure (1) is thus filled with 1,089 (33×33) light diffusion tubes (4).

In reality, this stacking is not inevitably optimal from the point of view of lighted volume, as simulations show that it is preferable to shift every other line by λ_{eff}+d/2. In this configuration (a hexagonal network) the culture enclosure (1) is then filled with 1,270 diffusion tubes (4).

More precisely, optimization of the “bath” of light (intensity dynamics and intensity) must be carried out using calculations. By setting the average luminous intensity in the bath and local variations in light intensity, the optimal surface of the diffusion tubes (4) can be determined for a given luminous power injected by each LED (2), and from hence the optimal diameter.

### Culture Medium Circulation System: Bubble Generator

Dynamic operation of the photobioreactor further supposes that at its bottom pressurized gas is advantageously injected (optionally with nutrients). This injection, notably through a device called a “sparger”, leads to the creation of a stream of bubbles which causes the biological liquid to rise. The photobioreactor thus advantageously comprises a bubble generation system (13) arranged at the bottom of the culture medium (3).

FIGS. 4 and 5 represent various geometries of the bubble sparging system (13) able to inject these bubbles in a controlled manner at the bottom of the culture medium (3).

Reactors that function according to this classic principle are called air-lift reactors. The principal flow of liquid, although oriented in the upward direction (then in the downward direction), leads the microalgae to “diffuse” transversely between the diffusion tubes (4). The microalgae by moving thusly collect a variable light, since in this direction the light decrease profile is exponential when moving away from the diffusion tubes (4). The microalgae thus receive average power at the wavelength λ_{eff}. The effectiveness of this “averaging” of the quantity of light received by each microalgae is that the diffusion time for a microalgae between the two diffusion tubes (4) is very short in relation to the life cycle of an alga, and preferably the time of ascent (or descent) of a microalgae in the culture enclosure (1).

Air-lift operation in general supposes an ascending flow of the culture medium (3) and obviously a downward flow. Fluid is injected at the bottom of the rising portion. Schematically, the culture enclosure (1) could be separated into two equivalent distinct parts, ascending and descending, the flow and the counter-flow being lighted by the same method of luminous fingers. Optimization of the liquid flow configuration can lead to other partitions of the culture enclosure (1) of the photobioreactor into N ascending blocks, M descending blocks, or to the use of tubes arranged at the bottom of the culture enclosure (1) and placed between the diffusion tubes (4).

It will be noted that the technology of the light diffusion elements (4) regardless of their geometry can in principle allow any shape of the culture enclosure (1) and not only parallelepiped or cylindrical.

Stacking the culture enclosures (1) is, however, easier in the case of parallelepipeds and makes it possible to optimize space. In the case of a cylindrical enclosure, the hydrodynamics of the ascending and descending flows, which are associated with concentric spargers (13) (see FIG. 5), are more delicate to manage.

In the inventive photobioreactor, it is shown that extending the interface between flows and counter-flows (ascending and descending) does not exceed the interval between two the planes of the diffusion tubes (4). This interface establishes itself naturally at the limit of the sparging zones.

Furthermore, as explained, the photobioreactor functions in “continuous” mode. Indeed, it is essential that microalgae density remains constant to maintain the same light penetration depth, therefore the concentration is stabilized by continuous sampling of the liquid, and counter-injecting part of the same quantity of water, optionally enriched with nutrients. This method is described in particular in the patent application FR1050015.

The photobioreactor can indeed comprise various regulation systems. Since such systems must function continuously for a given geometry, in particular related to diffusion element spacing, optimal algae density must be controlled in a stationary regime. This measurement involves the optical density of the biological environment.

Other parameters critical for optimizing microalgae growth can be the object of continuous measurements of pH, temperature, etc.

Generally these parameters will be set according to instructions that guarantee optimal operation.

### Photobioreactor Use

According to a second aspect, the invention relates to the use of a photobioreactor according to the first aspect of the invention to cultivate photosynthetic microorganisms, preferably microalgae.

Said use can be for applications related to energy (biofuel production), industry (pigment production), agri-food (omega-3 and polyunsaturated fatty acid production), pollution control (purification of carbon dioxide, NOx and/or SOx emissions) and even mass pharmaceuticals.

Another aspect of the invention relates as explained above to the use of a cylindrical or prismatic light diffusion element (4) coupled optically with a light source (2) so as to collect the photons emitted by the light source (2) and to return them by its lateral surface to illuminate the culture medium of a photobioreactor. The light diffusion element (4) can be the object of all of the embodiments described above.

### Numerical Example

### Parameters

Diffusion tubes (10 mm diameter);

Cubic enclosure (1) (1 m on each side);

LEDs (2) with a power of 10 W electrical or 2.5 W optical (650 nm wavelength);

Characteristic light penetration depth λ=3.8 mm (concentration of 10⁸ cells/ml);

Algae of the genus Nannochloris of unit mass 10⁻¹¹ g (biological mass of 1 g/l as a consequence), effectiveness threshold I_{eff}=50 μmol/^{m-2}/s⁻¹;

“Square” arrangement of the light tubes.

Considering that the diffusion tubes (4) have a length of 1 m, equal to the dimensions of the culture enclosure (1), a lateral surface of 314 cm² per diffusion tube (4) is calculated. The optical power injected being 2.5 W, considering as explained above that the diffusion tube (4) diffuses this power homogeneously, the luminous flux, i.e., the optical power transmitted to the medium per unit area, is 79.62 W/m² (on the tube surface), or 432 μmol/^{m-2}/s⁻¹.

This value must now be converted into moles of photons per m² per second. The energy of a photon is indeed related to its frequency (ν) (the inverse of its wavelength multiplied by the speed of light) by Planck's constant (h): E=hν. One mole of photons (6.02·10²³ photons, according to the Avogadro constant) at a wavelength of 650 nm thus has an energy of 173.9 kJ.

It is deduced therefrom that incidental the luminous flux is 432 μmol/^{m-2}/s⁻¹.

By using the formula mentioned in the description above, an effective length λ_{eff}=8.5 mm is obtained.

The square arrangement described above anticipates a variation of 2λ_{eff} between two successive diffusion tubes (4), and thus it is possible to place up to 1,369 (37×37) diffusion tubes (4) in the cubic enclosure (1).

The total lighting surface is thus 43 m², and the instantaneous electric consumption of the LEDs (2) is thus 13.7 kW, including 10.28 kWth to be dissipated.

The volume of culture medium (3) in the culture enclosure (1) corresponds to the total volume of 1 m³ less the volume of the 1369 diffusion tubes (4). It is 0.89 m³. The volume illuminated “effectively”, i.e., in a ring of width λ_{eff} around each diffusion tube (4), can be calculated as 0.67 m³.

On the basis of the principle that under continuous operation the mass of microalgae “effectively illuminated” doubles every 12 hours, a 0.94 kg/day production of microalgae for a photobioreactor with a 1 m³ culture enclosure is obtained, while consuming 329 kWh/d of electricity.

It is noted that in relation to lighting a 1 m² surface and a volume of 1 m³, the reactor's raw efficiency increased by a factor of 43, a number which takes into account that the hydrodynamics of the reactor is to be multiplied by a factor of 2, since here it is considered that the illuminated volume is to be multiplied by the factor λ_{eff}/A.

1. A photobioreactor intended for the culture of photosynthetic microorganisms, comprising at least one culture enclosure (1) intended to contain the microorganism culture medium (3) and at least one light source (2) outside the culture enclosure (1),
characterized in that it further comprises at least one cylindrical or prismatic light guide element (4) placed inside the culture enclosure (1), the light guide element (4) being coupled optically with the light source (2) so as to collect the photons emitted by the light source (2) and to transmit them to the culture medium (3) by its lateral surface, the light guide element (4) being a hollow element made of transparent material, at the end of which the light source (2) is placed, a semi-reflective layer (8) being arranged on the outside of the guide element (4), the thickness of the semi-reflective layer (8) decreasing with distance from the light source (2).

2. A photobioreactor of claim 1, characterized in that the semi-reflective layers (7, 8) are made of a metal or metal oxide material, with an optical index that is greater than the index of the material comprising the guide element (4).

3. A photobioreactor of claim 1, characterized in that the light guide element (4) is made of poly(methyl methacrylate).

4. A photobioreactor of claim 1, characterized in that the light source (2) is a quasi-punctual source, and the light guide element (4) is a tube.

5. A photobioreactor of one of claim 1, characterized in that the light source (2) is a linear source, and the light guide element (4) is a parallelepipoid.

6. A photobioreactor of claim 4, characterized in that the light source (2) is a light-emitting diode (LED) or a set of light-emitting diodes distributed quasi-punctually or in strips.

7. A photobioreactor of claim 6, characterized in that a convergent lens (5) is placed between the LED (2) and the light guide element (4).

8. A photobioreactor of claim 13, characterized in that an optical system (41) whose interior is reflective surrounds the LED (2).

9. A photobioreactor of claim 1, characterized in that the end of the light guide element (4) opposite the light source (2) is provided with a mirror (42).

10. A photobioreactor of claim 1, characterized in that the end of the light guide element (4) opposite the light source (2) is cone- or dome-shaped.

11. A photobioreactor of claim 1, characterized in that the external surface of the light guide element (4) is encapsulated in a protective sheath (10).

12. A photobioreactor of claim 11, characterized in that the light guide element (4) comprises a cleaning scraper (11) surrounding the sheath (10).

13. A photobioreactor of claim 1, comprising a cooling system (12) for the light sources (2).

14. A photobioreactor of claim 1, comprising a bubble generation system (13) at the bottom of the culture medium (3).

15. A method of using a photobioreactor of claim 1, to cultivate photosynthetic microorganisms, preferably microalgae.

16. A method of using a cylindrical or prismatic light guide element (4) coupled optically with a light source (2), the light guide element (4) being a hollow element made of transparent material, at the end of which the light source (2) is placed, a semi-reflective layer (8) being arranged on the outside of the guide element (4), the thickness of the semi-reflective layer (8) decreasing with distance from the light source (2) so as to collect the photons emitted by the light source (2) and to transmit them by its lateral surface to illuminate the culture medium of a photobioreactor.

17. A photobioreactor of claim 5, characterized in that the light source (2) is a light-emitting diode (LED) or a set of light-emitting diodes distributed quasi-punctually or in strips.

18. A photobioreactor of claim 14, characterized in that an optical system (41) whose interior is reflective surrounds the LED (2).
